(19) United States
(12) Patent Application Publication
(10) Pub. No.: US 2009/0326062 A1
(43) Pub. Date: Dec. 31, 2009

(54) PROCESS FOR PREPARING ENTACAPONE SUBSTANTIALLY FREE OF Z-ISOMER, SYNTHESIS INTERMEDIATES THEREOF AND A NEW CRYSTALLINE FORM

(75) Inventors: Francisco Eugenio Palomo Nicolau, Barcelona (ES); Andrés Molina Ponce, Barcelona (ES); Jordi Benet-Buchholz, Altafulla (ES); Lluis Sola Carandell, Altafulla (ES)

Correspondence Address: BARNES & THORNBURG LLP, 11 SOUTH MERIDIAN, INDIANAPOLIS, IN, 46204, US

(73) Assignee: Chemo Iberica, SA, Barcelona (ES)

(21) Appl. No.: 12/526,646

(22) PCT Filed: Feb. 13, 2008
(86) PCT No.: PCT/EP2008/051740

§ 371 Date: Aug. 11, 2009

(30) Foreign Application Priority Data
Feb. 13, 2007 (ES) P200700381

(51) Int. Cl. A61K 31/277; C07C 253/30; A61P 25/16

(52) U.S. Cl. 514/521; 558/401

(57) ABSTRACT

The present invention relates to a new method for obtaining Entacapone substantially free of Z-isomer from 3,4-dihydroxy-5-Nitrobenzaldehyde and N,N-Dimethylcyanoacetamide, or directly from a mixture of (E)- and (Z)-isomers of Entacapone, by formation of organic or inorganic salts, specially piperidine and sodium ones. A new crystalline form G of Entacapone can be obtained from this method in a fast, efficient, and simple way and substantially free of Z-isomer. Another object of the invention is a pharmaceutical composition comprising it.

### FIELD OF THE INVENTION

The present invention relates to a new process for preparing Entacapone substantially free of Z-isomer by formation of organic or inorganic salts as reaction intermediates.

The invention also relates to the new reaction intermediates formed in the process, particularly to Entacapone salts substantially free of Z-isomer.

The invention also relates to a new crystalline form G and a pharmaceutical composition comprising it.

### BACKGROUND OF THE INVENTION

Entacapone is an inhibitor of COMT (catechol-O-methyltransferase) indicated for the treatment of Parkinson's disease. For therapeutic purposes, the pure E isomer is used. The chemical name for Entacapone is (2E)-2-cyano-3-(3,4-dihydroxy-5-nitrophenyl)-N,N-diethyl-2-propenamide and its structure is shown below:

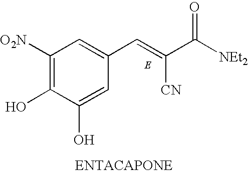

Entacapone was first disclosed in U.S. Pat. No. 4,963,590 as a regioisomeric mixture of two geometrical (E)- and (Z)-isomers. No techniques are discussed about the separation of said isomers.

Later, U.S. Pat. No. 5,131,950 disclosed a stable crystalline form, named Entacapone form A. According to said US patent, Entacapone was obtained as a mixture of two geometrical (E)- and (Z)-isomers in a ratio of 70-80% and 30-20%, respectively. Furthermore, the authors of this patent found out that Entacapone (E-isomer) exists in two polymorphic forms A and B; the (Z)-isomer as well as the form B showing to be unstable.

The process described in U.S. Pat. No. 5,131,950 for the preparation of Entacapone form A comprises the crystallization of crude Entacapone (Z/E) in an aliphatic carboxylic acid containing 1 or 2 carbon atoms and with a catalytic amount of HBr/HCl. Entacapone form A thus obtained, as described in U.S. Pat. No. 5,131,950 is a compound containing a maximum of 3% of the Z-isomer or other polymorphic forms.

On the other hand, it must be pointed out that there are other patent applications that describe other crystalline forms of Entacapone such as the international patent applications WO 05/066117-A, WO 05/063695-A, and WO 05/063696-A.

The object of the present invention is to provide a new process for preparing Entacapone substantially free of Z-isomer by formation of organic or inorganic salts, wherein it is also possible to obtain a new pure and stable crystalline form G of the Entacapone compound, which can be prepared in a simple, fast, and high-yielding way and which is characterizable and reproducible.

### BRIEF DESCRIPTION OF THE INVENTION

The aspect of the present invention is to provide a new process for preparing Entacapone substantially free of Z-isomer by formation of organic or inorganic salts as reaction intermediates.

Another aspect of the present invention is the new crystalline form G obtained from the above indicated new process, and the pharmaceutical composition comprising it.

Thus, another aspect of the present invention is the synthesis intermediates resulting from the process for preparing Entacapone substantially free of Z-isomer. In particular, still another object of the present invention is the Entacapone salts obtained as synthesis intermediates.

### DETAILED DESCRIPTION OF THE INVENTION

According to the first, second, and third aspects of the present invention, a new process for preparing Entacapone substantially free of Z-isomer by formation of organic or inorganic salts as reaction intermediates is provided. In turn, a new polymorphic form G of Entacapone is obtained under certain conditions.

According to the first aspect of the invention, a process for preparing Entacapone of formula (I) is provided herein:

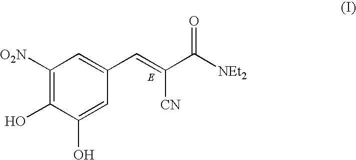

substantially free of Z-isomer comprising the following steps:

i) reaction of an Entacapone mixture (E/Z) (II) with an organic or inorganic base in a suitable solvent in order to provide an Entacapone salt of formula (III) enriched in the E-isomer:

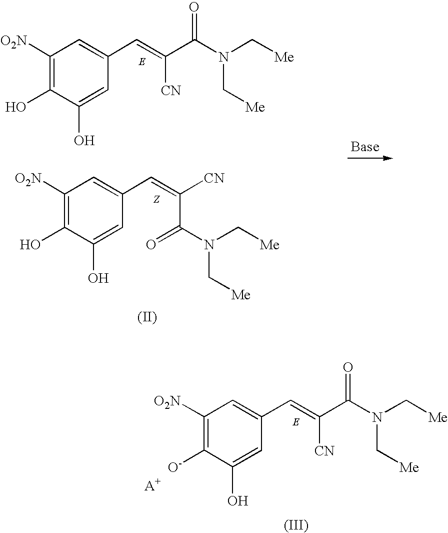

wherein A⁺ is the protonated base or the cation of the base, whether the base used is organic or inorganic, respectively;

ii) reaction of the Entacapone salt of formula (III) enriched in the E-isomer with an acid in a suitable solvent in order to obtain (E)-2-cyano-3-(3,4-dihydroxy-5-nitrophenyl)-N,N-diethyl-2-propenamide (Entacapone) substantially free of Z-isomer of formula (I):

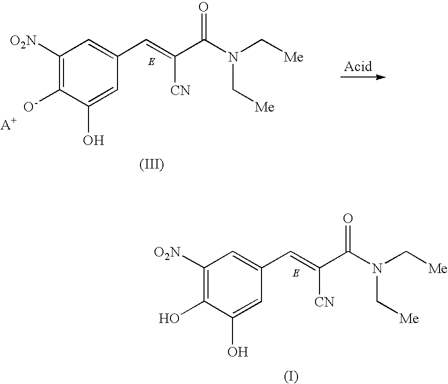

In general, when it comes to an Entacapone mixture (Z/E) with a base in a suitable solvent, the resulting mixture has been observed to be enriched in the E-isomer, particularly when the Entacapone salt is precipitated into the reaction medium. This surprising effect allows, by means of a base, to transform the Z-isomer into the E-isomer by the formation of the corresponding Entacapone salt.

This discovery contrasts with what is described in U.S. Pat. No. 5,131,950, where its authors explain that the Z-isomer can be easily converted into the E-isomer under the influence of acids through the crystallization of crude Entacapone (Z/E) in an aliphatic carboxylic acid with a catalytic amount of HBr/HCl.

Although this invention is not related to a specific theory to explain how the Z-isomer is converted into the E-isomer of Entacapone, the authors of the present invention postulate that this transformation could result from the unlocated anion through the conjugated system with double bonds in the molecule, as shown in the figure.

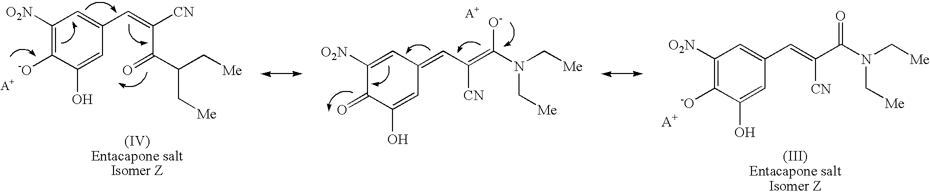

The crude Entacapone (Z/E), used in the method as a starting product, can be obtained for instance according to the method described in U.S. Pat. No. 4,963,590 or can be carried out partially the example 3.1 of the patent application WO 2005/063695-A, without performing the treatment with HBr/AcOH.

Thus, in an alternative embodiment of the invention, the Entacapone mixture (E/Z) can be generated in situ through the reaction of the 3,4-dihydroxy-5-Nitrobenzaldehyde compound of formula (V) with the N,N-dimethylcyanoacetamide of formula (VI) in the presence of a base in a suitable solvent, preferably an alcoholic solvent:

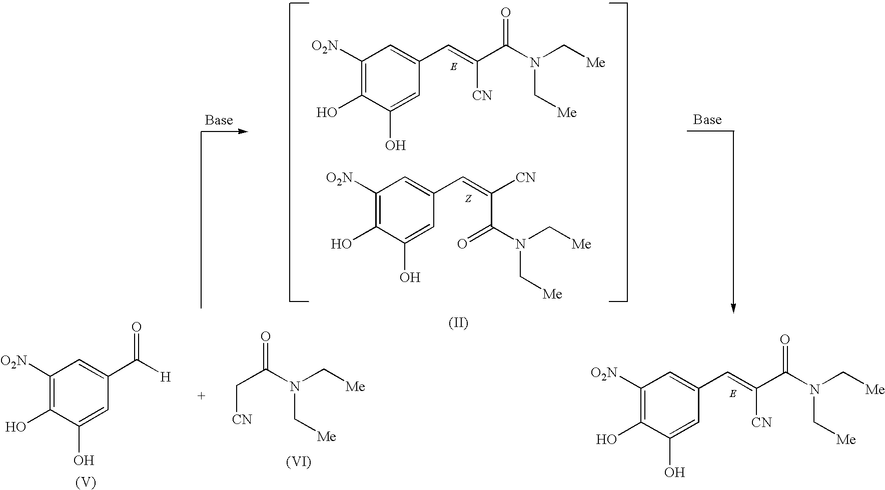

so that the resulting Entacapone mixture (E/Z) (II) is converted in the reaction medium into an Entacapone salt of formula (III) enriched in the E-isomer, as defined above.

Surprisingly, the authors of the present invention have found that Entacapone substantially free of Z-isomer can be obtained by the formation of organic and inorganic salts as Entacapone reaction intermediates.

The base used can be organic or inorganic. In the case of an organic base, it is preferably selected from the group consisting of piperidine, piperazine, and morpholine, more preferably, piperidine. In the case of an inorganic base, it is preferably selected from hydroxides of alkali or alkaline earth metals, more preferably, sodium hydroxide.

The amount of base used is between 1 to 3 moles, preferably 1.5 moles, for each mol of the Entacapone mixture (Z/E) (II), when based in crude containing an Entacapone mixture (Z/E), or for each mol of the compound (V), when the Entacapone mixture (Z/E) is generated in situ through the reaction of the 3,4-dihydroxy-5-Nitrobenzaldehyde compound of formula (V) with the N,N-Dimethylcyanoacetamide of formula (VI).

The solvent used is preferably a chain C₁₋₄ alcohol. More preferably, it is selected from isopropanol and ethanol.

The Entacapone salt of formula (III) obtained in step i) as described above is converted into Entacapone substantially free of Z-isomer through the reaction with an acid. This transformation can be performed after the isolation of the Entacapone salt by filtration, or it can be performed in situ without the isolation of said salt.

In one embodiment of the invention, Entacapone substantially free of Z-isomer can be obtained from a one pot reaction, where steps i) and ii) are performed without isolation.

In another preferred embodiment of the invention, the Entacapone salt (III) is isolated from the reaction medium by filtration and reacted with an acid within a solvent or solvent mixture. Preferably, the Entacapone salt is suspended in a chain C₁-C₄ alcohol, more preferably in isopropanol or ethanol, and reacted with an acid.

The acid used can be organic or inorganic. In the case of an inorganic acid, hydrochloric acid is preferably used. In the case of an organic acid, p-toluenesulfonic acid is preferably used.

The amount of acid is between 1 to 2 moles, preferably between 1.0 to 1.5 moles, for each mol of Entacapone salt of formula (III).

In the present invention, “substantially free of Z-isomer” means that the amount of Z-isomer is not higher than 0.5%, preferably not higher than 0.1%, determined by HPLC.

In one embodiment of the present invention, piperidine is the organic base used. Therefore, the piperidine salt of Entacapone is obtained as the reaction intermediate.

In another preferred embodiment of the present invention, sodium hydroxide is the inorganic base used. Therefore, the sodium salt of Entacapone is obtained.

Another object of the present invention and one preferred embodiment of the process for preparing Entacapone substantially free of Z-isomer according to the invention is the method for preparing a new crystalline form G of Entacapone, from the following steps:

a) preparing a suspension of an Entacapone salt of formula (III) enriched in the E-isomer as obtained in step i) defined above, in a C₁₋₄ alcohol, preferably an isopropyl alcohol, and then

b) adding a diluted inorganic acid, preferably hydrochloric acid of 35% of strength, in said suspension at a temperature from 15 to 35° C., preferably from 20 to 30° C.

Surprisingly, a new crystalline form of Entacapone (form G) is obtained under these conditions. The new crystalline form G of Entacapone is obtained in a stable form, with high yield and purity. These characteristics make this new polymorphic form suitable for the development of a pharmaceutical product.

Preferably, the new crystalline form G of Entacapone is obtained from the piperidine salt of Entacapone (IIIa) or the sodium salt of Entacapone (IIIb).

Another object of the present invention is to provide a pharmaceutical composition comprising the crystalline form of Entacapone form G in combination with one or more excipients or other pharmaceutically acceptable auxiliary agents.

The new crystalline form G of Entacapone was characterized.

For the record of the X-ray powder diffraction pattern, a diffractometer has been used with the following characteristics:

### PANALYTICAL XPERT PRO

Copper tube, at 40 kV and 40 mA.

### X CELERATOR Detector

Angular scanning of 2-45° (2 theta). Step size: 0.050°.
Scanning step time: 46.08 s.
Graphite monochromator. Automatic slit.
Revolving sample holder with spinner.

The interplanar d-spaces and the relative intensities that characterize the new crystalline form G of Entacapone are shown in Table 1.

TABLE 1
X-ray diffraction peaks

| | | Relative intensity |
|---|---|---|
| 2θ | D | (%) |
| 5.92 | 14.93 | 100.00 |
| 13.43 | 6.59 | 3.33 |
| 13.77 | 6.43 | 5.16 |
| 14.07 | 6.29 | 5.53 |
| 14.68 | 6.03 | 13.68 |
| 14.98 | 5.91 | 17.49 |
| 17.81 | 4.98 | 16.80 |
| 18.20 | 4.87 | 25.59 |
| 20.27 | 4.38 | 13.09 |
| 21.53 | 4.13 | 3.23 |
| 22.58 | 3.94 | 1.87 |
| 23.43 | 3.80 | 6.69 |
| 23.81 | 3.73 | 9.48 |
| 25.04 | 3.56 | 9.44 |
| 25.48 | 3.49 | 7.56 |
| 26.61 | 3.35 | 12.97 |
| 26.94 | 3.31 | 13.02 |
| 27.72 | 3.22 | 4.82 |
| 28.35 | 3.15 | 5.33 |
| 29.23 | 3.05 | 11.95 |
| 29.73 | 3.00 | 4.52 |
| 30.16 | 2.96 | 4.38 |
| 30.91 | 2.89 | 2.49 |
| 31.58 | 2.83 | 2.10 |
| 32.80 | 2.73 | 3.60 |
| 34.16 | 2.62 | 4.21 |

The Infra Red spectrum was obtained by grinding the KBr and sample mixture, with a sample content of 1% of strength, in an agate mortar by reflectance. The typical peaks by IR that characterize the new crystalline form G of Entacapone are:

IR (cm⁻¹): 3160, 3103, 2998, 2986, 2939, 2880, 2740, 2209, 1613, 1592, 1541, 1503, 1479, 1461, 1446, 1366, 1351, 1308, 1280, 1244, 1236, 1217, 1197, 1172, 1152, 1142, 1097, 1083, 1071, 1021, 995, 946, 925, 903, 885, 865, 805, 787, 764, 727, 683, 645, 609, 555.

The purity of the resulting Entacapone was determined by HPLC:

### Column: Inertsil ODS-3V, 250×4.6 mm, 5 μm

### Wavelength: 304 nm.

Flow rate: 1.0 ml/min.

### Temperature: 30° C.

Buffer: 0.1% aqueous solution of trifluoroacetic acid.
Mobile phase: gradient.

| | Elapsed minutes | | | | |
|---|---|---|---|---|---|
| | 0 | 30 | 35 | 36 | 40 |
| % buffer | 70 | 30 | 30 | 70 | 70 |
| % acetonitrile | 0 | 70 | 70 | 30 | 30 |

Sample preparation: 0.2 mg/ml dissolved in acetonitrile.
Retention time: Z-isomer (13.4 min); E-isomer (14.3 min).

The following examples serve to illustrate the invention without limiting the objects defined in the attached claims.

### EXAMPLES

### Example 1

### Process for Preparing Entacapone Substantially Free Of Z-Isomer from 3,4-Dihydroxy-5-Nitrobenzaldehyde (V) and N,N-Dimethylcyanoacetamide (VI), Using an Organic Base of Piperidine to Provide the Piperidine Salt of Entacapone as Synthesis Intermediate

a) Method for Obtaining Piperidine Salt of Entacapone (IIIa)

A mixture of 3,4-dihydroxy-5-Nitrobenzaldehyde (70 g; 382 mmole), N,N-Diethylcyanoacetamide (107 g; 764 mmole), piperidine (56.6 ml; 573 mmole), and acetic acid (32.8 ml; 573 mmole) in isopropanol (700 ml) is heated at reflux during approximately 3 hours. The resulting dissolution is cooled to room temperature and the resulting precipitate is kept in stirring at this temperature overnight. Finally, it is cooled at 0-5° C., filtered off and washed with isopropanol (140 ml). The resulting product is dried at 40° C. in a vacuum oven to provide 119 g (79.7% yield) of an orange solid (m.p.=152-4° C.; HPLC purity=98.0% (Z-isomer=0.94%)).

IR (cm⁻¹): 3190, 3038, 2975, 2828, 2723, 2547, 2201, 1631, 1607, 1542, 1480, 1439, 1387, 1357, 1318, 1265, 1221, 1187, 1176, 1156, 1074, 1018, 948, 866, 834, 802, 782, 681, 638, 607, 562.

¹H-NMR (500 MHz, CD₃OD): 7.94 (d, J=2.4 Hz, 1H); 7.65 (d, J=2.4 Hz, 1H); 7.47 (s, 1H); 3.56 (q, J=6.6 Hz; 4H); 3.35-3.16 (m, 4H); 1.84-1.80 (m, 4H); 1.74-1.71 (m, 2H); 1.29 (t, J=6.6 Hz, 6H).

Analysis. Calculated for C₁₄H₁₄N₃O₅.C₅H₁₂N: C, 58.45; H, 6.17; N, 14.35. Found: C, 58.19; H, 6.52; N, 14.27.

b) Method for Obtaining Entacapone Substantially Free of Z-Isomer from the Piperidine Salt of Entacapone (Form G of Entacapone)

A dissolution comprising a mixture of water (1200 ml) and 35% aq. HCl is added to a suspension of the piperidine salt of Entacapone obtained in a) (119 g; 305 mmole) in isopropanol (600 ml), keeping the temperature from 20 to 30° C. (29.8 ml; 335 mmole). The resulting precipitate is cooled at 0-5° C., filtered off and washed with isopropanol/water (80 ml: 160 ml), and finally, with water (240 ml). The resulting product is dried at 40° C. in a vacuum oven to provide 84.8 g (yield=91.1%) of an orange solid (m.p.=162.4-163.5° C.; HPLC purity=99.8% (Z-isomer=0.05%)).

### Example 2

### Method for Obtaining Entacapone Substantially Free of Z-Isomer from Crude Entacapone (Z/E), Using an Organic Base of Piperidine to Provide the Piperidine Salt of Entacapone as Synthesis Intermediate

a) Method for Obtaining Piperidine Salt of Entacapone (IIIa)

Piperidine (6.26 g; 73.5 mmole) is added to a suspension of Entacapone (E-isomer=75%; Z-isomer=25%) (12.5 g; 40.9 mmole) in isopropanol (150 ml) at room temperature. The mixture is stirred for approximately 2 hours, obtaining an abundant precipitate. Finally, it is cooled at 0-5° C. for approximately 2 hours and the resulting precipitate is filtered off and washed with cold isopropanol (20 ml). The resulting product is dried at 40° C. in a vacuum oven to provide 14.2 g (yield=88.8%) of an orange solid (m.p.=152-4° C. (decomp.); (Z-isomer=1.3%)).

b) Method for Obtaining Entacapone Substantially Free of Z-Isomer from the Piperidine Salt of Entacapone (Form G of Entacapone)

Entacapone substantially free of Z-isomer product can be obtained from the piperidine salt of Entacapone obtained in a), under the conditions in the example 1b.

### Example 3

### Method for Obtaining Entacapone Substantially Free of Z-Isomer from Crude Entacapone (Z/E), Using an Inorganic Base of Sodium Hydroxide to Provide the Sodium Salt of Entacapone as Synthesis Intermediate

a) Method for Obtaining Sodium Salt of Entacapone (IIIb)

30% aq. NaOH is added to a suspension of Entacapone (E-isomer=69%; Z-isomer=31%) (15.15 g; 40.9 mmole) in ethanol (100 ml) at room temperature (8.73 g; 65.5 mmole). The mixture is stirred at room temperature and the resulting precipitate is kept in stirring at this temperature overnight. Finally, it is cooled at 0-5° C. for approximately 2 hours and filtered off and washed with cold ethanol (20 ml). The resulting product is dried at 40° C. in a vacuum oven to provide 14.13 g (yield=87.1%) of a red solid (m.p.=260-4° C. (decomp.); (Z-isomer=1.80%)).

IR (cm⁻¹): 3317, 2990, 2201, 1641, 1592, 1538, 1475, 1460, 1443, 1390, 1350, 1265, 1213, 1163, 1102, 1087, 1070, 1017, 996, 944, 876, 863, 827, 799, 786, 742, 625, 602, 564.

¹H-NMR (500 MHz, CD₃OD): 7.81 (dd, J=0.7, 2.6 Hz, 1H); 7.37 (s, 1H); 7.36 (dd, J=0.4, 2.6 Hz, 1H); 3.38 (q, J=7.1 Hz, 4H); 1.13 (t, J=7.1 Hz, 6 Hz).

Analysis. Calculated for C₁₄H₁₄N₃O₅.Na: C, 51.38; H, 4.31; N, 12.84. Found: C, 50.93; H, 4.29; N, 12.71.

b) Method for Obtaining Entacapone Substantially Free of Z-Isomer from the Sodium Salt of Entacapone (Form G of Entacapone)

Entacapone substantially free of Z-isomer product can be obtained from the sodium salt of Entacapone obtained in a), under the conditions in the example 1b.

What is claimed is:

1-29. (canceled)

30. A method for obtaining Entacapone of formula (I) 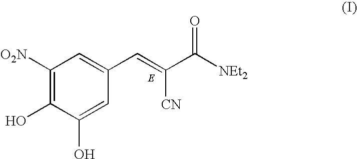
wherein the amount of Z-isomer is not higher than 0.5%, which comprises the following steps:
i) reacting an Entacapone mixture (E/Z) (II) with an organic or inorganic base in a suitable solvent in order to provide an Entacapone salt of formula (III) enriched in the E-isomer: 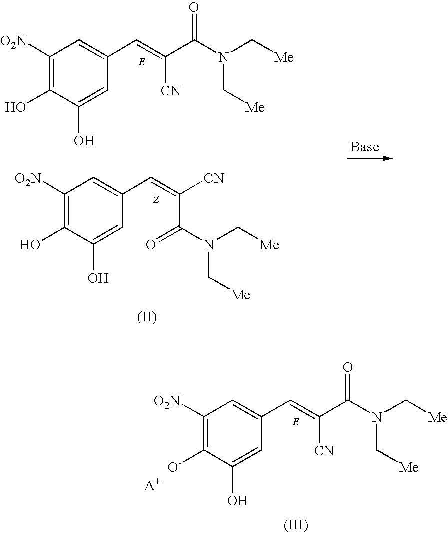
wherein A⁺ is the protonated base or the cation of the base, whether the base used is organic or inorganic, respectively; allowing said base to transform the Z-isomer into the E-isomer by the formation of the corresponding salt; and
ii) reacting the Entacapone salt of formula (III) enriched in the E-isomer with an acid in a suitable solvent in order to obtain (E)-2-cyano-3-(3,4-dihydroxy-5-nitrophenyl)-N,N-diethyl-2-propetamide (Entacapone) of formula (I), wherein the amount of Z-isomer is not higher than 0.5%:

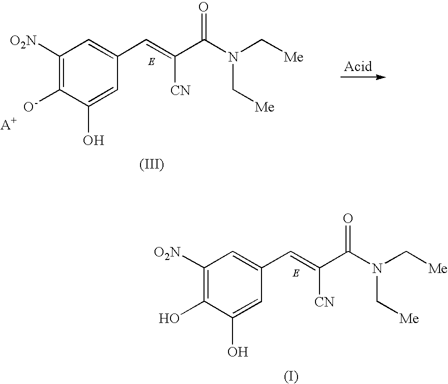

31. A method according to claim 30, characterized in that said organic base is selected from the group consisting of piperidine, piperazine, and morpholine.

32. A method according to claim 31, wherein said organic base is piperidine.

33. A method according to claim 30, characterized in that said inorganic base is selected from hydroxides of alkali or alkaline earth metals.

34. A method according to claim 33, wherein said inorganic base is sodium hydroxide.

35. A method according to claim 30, characterized in that said base is present in an amount of 1.5 moles for each mol of said Entacapone mixture (Z/E) (II).

36. A method according to claim 30, characterized in that said solvent is selected from a chain C₁₋₄ alcohol, or mixture of said.

37. A method according to claim 36, wherein said solvent is selected from isopropanol and ethanol.

38. A method according to claim 30, characterized in that in step ii) said acid is an organic or inorganic acid.

39. A method according to claim 38, wherein said organic acid is p-toluenesulfonic acid.

40. A method according to claim 38, wherein said inorganic acid is hydrochloric acid.

41. A method according to claim 30 characterized in that in step ii) said acid is present in an amount between 1 to 2 moles for each mol of Entacapone salt of formula (III) enriched with E-isomer.

42. A method according to claim 41, characterized in that said acid is present in an amount between 1.0 to 1.5 moles for each mol of Entacapone salt of formula (III) enriched with E-isomer.

43. A method according to claim 30, characterized in that the Entacapone salt (III) enriched with E-isomer obtained in step i) is isolated, optionally by filtration, before performing step ii).

44. A method according to claim 30, characterized in that steps i) and ii) are performed in a one pot reaction.

45. A method according to claim 30, characterized in that said Entacapone salt (III) enriched with E-isomer obtained in step i) is selected from the piperidine salt of Entacapone and the sodium salt of Entacapone.

46. Sodium salt of Entacapone (IIIb).

47. Crystalline form G of Entacapone, characterized by an X-ray powder diffraction pattern with the following typical peaks:

| 2θ | D |
|---|---|
| 5.92 | 14.93 |
| 13.43 | 6.59 |
| 13.77 | 6.43 |
| 14.07 | 6.29 |
| 14.68 | 6.03 |
| 14.98 | 5.91 |
| 17.81 | 4.98 |
| 18.20 | 4.87 |
| 20.27 | 4.38 |
| 21.53 | 4.13 |
| 22.58 | 3.94 |
| 23.43 | 3.80 |
| 23.81 | 3.73 |
| 25.04 | 3.56 |
| 25.48 | 3.49 |
| 26.61 | 3.35 |
| 26.94 | 3.31 |
| 27.72 | 3.22 |
| 28.35 | 3.15 |
| 29.23 | 3.05 |
| 29.73 | 3.00 |
| 30.16 | 2.96 |
| 30.91 | 2.89 |
| 31.58 | 2.83 |
| 32.80 | 2.73 |
| 34.16 | 2.62 |

48. A crystalline form G according to claim 47, characterized by the following Infra Red spectrum peaks: 3160, 3103, 2998, 2986, 2939, 2880, 2740, 2209, 1613, 1592, 1541, 1503, 1479, 1461, 1446, 1366, 1351, 1308, 1280, 1244, 1236, 1217, 1197, 1172, 1152, 1142, 1097, 1083, 1071, 1021, 995, 946, 925, 903, 885, 865, 805, 787, 764, 727, 683, 645, 609, 555.

49. A method for obtaining the crystalline form G of Entacapone comprising:
a) preparing a suspension of an Entacapone salt of formula (III) enriched in the E-isomer obtained according to claim 30 in a C₁₋₄ alcohol, and
b) adding a diluted inorganic acid in said suspension prepared in step a) at a temperature from 15 to 35° C.

50. A method according to claim 49, characterized in that said Entacapone salt of formula (III) is selected from the piperidine salt of Entacapone (IIIa) and the sodium salt of Entacapone (IIIb).

51. A method according to claim 49, characterized in that said C₁₋₄ alcohol is isopropyl alcohol.

52. A method according to claim 49, characterized in that said inorganic acid is hydrochloric acid of 35% of strength.

53. A pharmaceutical composition comprising the crystalline form G of Entacapone according to claim 47 as well as at least one excipient and/or other pharmaceutically acceptable auxiliary agents.

54. A method for treating a physiological disorder associated with COMT comprising administering to a human in need thereof an effective amount of the crystalline form G of Entacapone to said human.

55. The method of claim 54 wherein said disorder is Parkinson's disease.
